(19) United States
(12) Patent Application Publication
(10) Pub. No.: US 2007/0276497 A1
(43) Pub. Date: Nov. 29, 2007

(54) Surgical spacer

(75) Inventor: Kent M. Anderson, Memphis, TN (US)

Correspondence Address: HESLIN ROTHENBERG FARLEY & MESITI P.C., 5 COLUMBIA CIRCLE, ALBANY, NY, 12203, US

(73) Assignee: SDGI Holdings. Inc., Wilmington, DE (US)

(21) Appl. No.: 11/438,940

(22) Filed: May 23, 2006

(51) Int. Cl. A61F 2/44

(52) U.S. Cl. 623/17.12

(57) ABSTRACT

An interspinous spacer for placement between adjacent spinous processes includes a flexible, fillable container (e.g., a bag or balloon) for containing a material that is compressible during end use, for example, silicone after curing. The container is impermeable to the material it will be filled with. A fabric mesh, for example, made of PET fabric, provides structure for and containment of the container. The material can be injected into the container through an optional conduit, for example, a one-way valve.

### CROSS-REFERENCE TO RELATED APPLICATIONS/PATENTS

This application contains subject matter which is related to the subject matter of the following applications, each of which is assigned to the same assignee as this application and filed on the same day as this application. Each of the below listed applications is hereby incorporated herein by reference in its entirety:
“Surgical Spacer with Shape Control,” by Lange et al. (Attorney Docket No. P23190.00).
“Systems and Methods for Adjusting Properties of a Spinal Implant,” by Lange et al. (Attorney Docket No. P23186.00); and

### TECHNICAL FIELD

The present invention generally relates to surgical spacers for spacing adjacent body parts. More particularly, the present invention relates to surgical spacers having a flexible container for containing a material that is compressible during end use, the container being substantially impermeable to the material, and a structure for at least part of the container when containing the material, and methods of surgical spacing using such surgical spacers.

### BACKGROUND OF THE INVENTION

The human spine is a biomechanical structure with thirty-three vertebral members, and is responsible for protecting the spinal cord, nerve roots and internal organs of the thorax and abdomen. The spine also provides structural support for the body while permitting flexibility of motion. A significant portion of the population will experience back pain at some point in their lives resulting from a spinal condition. The pain may range from general discomfort to disabling pain that immobilizes the individual. Back pain may result from a trauma to the spine, the natural aging process, or the result of a degenerative disease or condition.

Procedures to address back problems sometimes require correcting the distance between spinous processes by inserting a device (e.g., a spacer) therebetween. The spacer, which is carefully positioned and aligned within the area occupied by the interspinous ligament, after removal thereof, is sized to position the spinous processes in a manner to return proper spacing thereof.

Dynamic interspinous spacers are currently used to treat patients with a variety of indications. Essentially, these patients present a need for distraction of the posterior elements (e.g., the spinal processes) using a mechanical device. Current clinical indications for the device, as described at SAS (Spine Arthroplasty Society) Summit 2005 by Guizzardi et al., include stenosis, disc herniation, facet arthropathy, degenerative disc disease and adjacent segment degeneration.

Marketed interspinous devices include rigid and flexible spacers made from PEEK, titanium or silicone. Clinical success with these devices has been extremely positive so far as an early stage treatment option, avoiding or delaying the need for lumbar spinal fusion. However, all devices require an open technique to be implanted, and many require destroying important anatomical stabilizers, such as the supraspinous ligament.

Current devices for spacing adjacent interspinous processes are preformed, and are not customizable for different sizes and dimensions of the anatomy of an interspinous area of an actual patient. Instead, preformed devices of an approximately correct size are inserted into the interspinous area of the patient. Further, the stiffness or flexibility of the devices must be determined prior to the devices being inserted into the interspinous area.

Thus, a need exists for improvements to surgical spacers, such as those for spacing adjacent interspinous processes.

### SUMMARY OF THE INVENTION

Briefly, the present invention satisfies the need for improvements to surgical spacers by providing a flexible container that is fillable in situ, together with at least a partial structure for the flexible container. In this way, the spacer is customizable, depending on the amount of material the container is filled with, allowing for conformity to the patient's anatomy, as well as being less invasive. An optional conduit coupled to the container allows for filling of the container, for example, by injecting the material.

The present invention provides in a first aspect, a surgical spacer. The surgical spacer comprises a flexible container for containing a material that is compressible during end use, wherein the container is substantially impermeable to the material. The surgical spacer further comprises a structure for at least part of the container when containing the material.

The present invention provides in a second aspect, a method of surgically spacing adjacent body parts. The method comprises providing a surgical spacer, comprising a flexible container for containing a material that is compressible during end use, wherein the container is fillable and substantially impermeable to the material. The spacer further comprises a structure for at least part of the container when containing the material. The method further comprises implanting the surgical spacer between adjacent body parts, and filling the container with the material.

The present invention provides in a third aspect, an interspinous spacer. The interspinous spacer comprises a flexible container for containing an injectable curable material that is compressible during end use, wherein the container is substantially impermeable to the injectable curable material. The interspinous spacer further comprises a structural mesh for at least part of the container when containing the injectable curable material, wherein the structural mesh is shaped to fit between adjacent spinous processes, and a conduit coupled to the container for accepting the injectable curable material.

The present invention provides in a fourth aspect, a method of spacing adjacent spinous processes. The method comprises providing an interspinous spacer. The interspinous spacer comprises a flexible container for containing an injectable curable material that is compressible during end use, wherein the container is impermeable to the injectable material. The spacer further comprises a structural mesh for at least part of the container when containing the injectable curable material, wherein the structural mesh is shaped to fit between adjacent spinous processes, and a valve coupled to the container for accepting the injectable curable material. The method further comprises implanting the interspinous spacer between adjacent spinous processes, and injecting the injectable curable material into the container through the valve.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 depicts adjacent vertebrae of the lumber region of a human spinal column.

FIG. 2 depicts a more detailed view of a portion of a human spinal column including the vertebrae of FIG. 1.

FIG. 3 depicts the spinal column portion of FIG. 2 after implantation and filling of one example of an interspinous spacer in accordance with an aspect of the present invention.

FIG. 4 is a partial cut-away view of one example of an unfilled surgical spacer with the container outside the structure, in accordance with an aspect of the present invention.

FIG. 5 depicts an example of a surgical spacer with integrated container and structure, in accordance with an aspect of the present invention.

FIG. 6 is a cross-sectional view of one example of a surgical spacer with external container, in accordance with an aspect of the present invention.

FIG. 7 depicts one example of the construction of a fabric jacket for use with one example of a surgical spacer, in accordance with an aspect of the present invention.

### DETAILED DESCRIPTION OF THE INVENTION

A surgical spacer of the present invention can be formed in situ during a procedure. The spacer includes two basic aspects: a flexible container, and a structure for at least part of the container. The flexible container can be filled or injected though an optional conduit after placement. Further, the structure may be folded in some aspects. Together with an unfilled container, in some aspects, the spacer can create a smaller footprint during implantation, which is less invasive, requires less tissue disruption for creating access for implantation, and allows the spacer to conform to the patient's anatomy. Once filled, the structure provides support and containment for the container, reducing the chances of complications like bulging of the container.

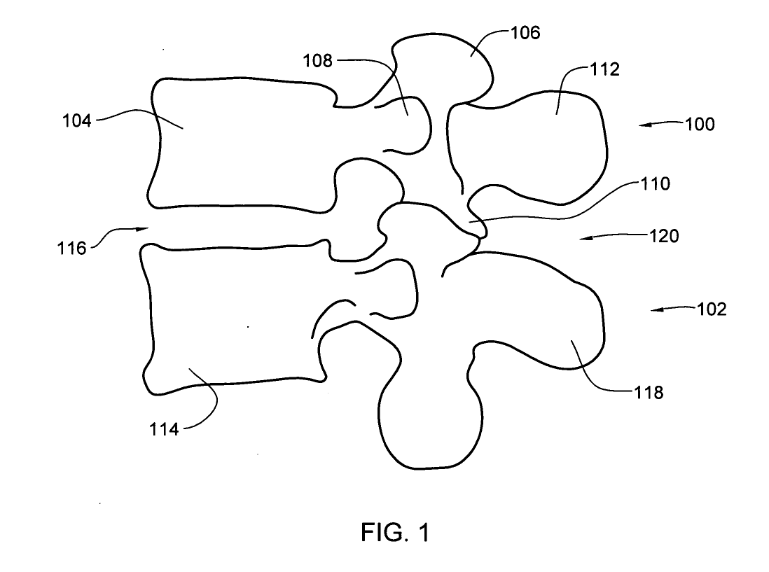

FIG. 1 depicts adjacent vertebrae 100, 102 of the lumbar region of a human spinal column. As known in the art, each vertebrae comprises a vertebral body (e.g., vertebral body 104), a superior articular process (e.g., superior articular process 106), a transverse process (e.g., transverse process 108), an inferior articular process (e.g., inferior articular process 110), and a spinous process (e.g., spinous process 112). In addition, between vertebral bodies 104 and 114 is a space 116 normally occupied by an intervertebral disc (see FIG. 2), and between spinous processes 112 and 118 is a space 120 normally occupied by an interspinous ligament (see FIG. 2).

FIG. 2 depicts the vertebrae of FIG. 1 within an area 200 of the lumbar region of a human spine. As shown in FIG. 2, spinous processes 112 and 118 are touching and pinching interspinous ligament 202, calling for spacing of the spinous processes.

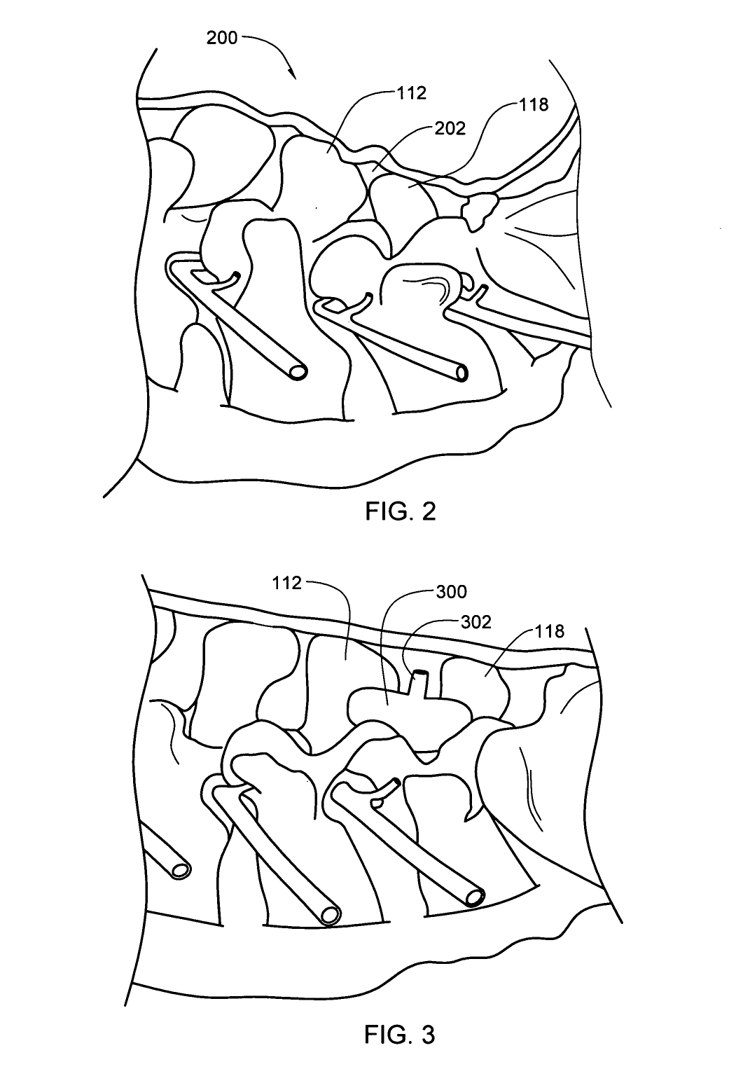

FIG. 3 depicts spinous processes 112 and 118 after spacing with an interspinous spacer 300 in accordance with one aspect of the present invention. As shown in FIG. 3, interspinous ligament 202 has been removed in a conventional manner prior to insertion of spacer 300. Although shown in its filled state, in this example, spacer 300 is implanted in its unexpanded state, as described more fully below. The spacer is filled with a material described below through a conduit 302 after implantation. For example, the material may be injected into the spacer through the conduit (e.g., a one-way valve). Prior to implantation and filling, measurement of the space between the interspinous processes and determination of the spacer size and desired amount of filling can be performed. Conventional methods can be used, such as, for example, the use of templates, trials, distractors, scissor-jacks or balloon sizers.

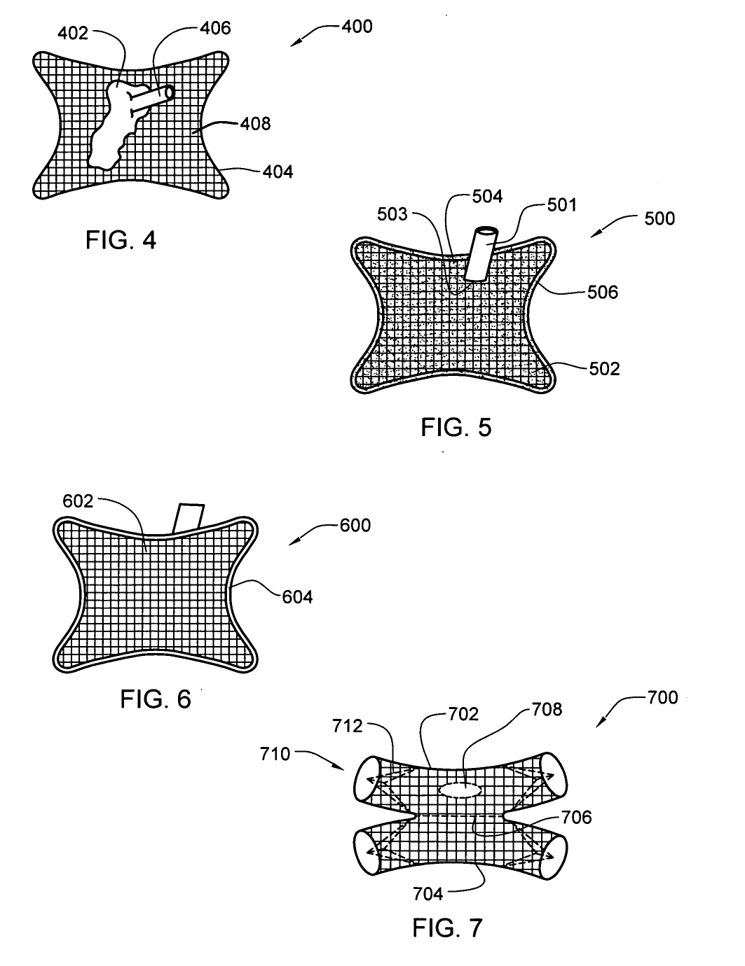

FIG. 4 depicts a partially cut-away view of one example of a spacer 400, in accordance with one aspect of the present invention. As shown in FIG. 4, the spacer comprises an unfilled container 402 inside a structure 404. Preferably, the container is in an evacuated state during implantation and prior to being filled. Where a valve (e.g., a one-way valve) is coupled to the container, the container is preferably evacuated prior to or during the process of coupling the valve thereto. In the present example, the structure is outside the container. However, as will be described in more detail below, the container can be outside the structure, or the container and structure can be integrated. In addition, although the structure is shown to be roughly H-shaped to fit between adjacent spinous processes, the structure can have any shape necessary for the particular surgical application. For example, the structure could instead have a roughly cylindrical shape to replace an intervertebral disc. As another example, the structure could be spherically or elliptically shaped to replace part of the intervertebral disc, for example, the nucleus pulpous, leaving the rest of the disc intact. Further, although the structure is shown enveloping the container, the structure could be for only a portion of the container, depending on the particular application. For example, it may be desired to prevent bulging of the container only in a particular area. Coupled to the container is an optional conduit 406 for accepting a material that is compressible during end use. The structure provides support for and containment of the container when filled.

The container is flexible and substantially impermeable to the material it will be filled with. However, depending on the application, the container may be permeable to other materials, for example, it may be air and/or water permeable. In the present example, the container takes the form of a bag or balloon, but can take other forms, so long as flexible and substantially impermeable to the material it will be filled with. Thus, the container must be substantially impermeable to the filling material, for example, in a liquid state during filling and prior to curing. Examples of container materials include silicone, rubber, polyurethane, polyethylene terephthalate (PET), polyolefin, polycarbonate urethane, and silicone copolymers.

Conduit 406 accepts the material being used to fill the container. Preferably, the conduit comprises a one-way valve, however, a two-way valve is also contemplated, as another example. Also preferably, the conduit is constructed to be used with a delivery system for filling the container, such as, for example, a pressurized syringe-type delivery system. However, the delivery system itself forms no part of the present invention. As noted above, the conduit is optional. Other examples of how to fill the container comprise the use of a self-sealing material for the container, or leaving an opening in the container that is closed (e.g., sewn shut) intraoperatively after filling. Using a curable material to fill the container may also serve to self-seal the container.

In use, the container is filled with a material that is compressible during end use. The compressibility characteristic ensures that the material exhibits viscoelastic behavior and that, along with the structure, the spacer can accept compressive loads. Of course, the degree of compressibility will depend on the particular application for the surgical spacer. For example, if a spacer according to the present invention is used between adjacent spinous processes, the spacer would need to accept compressive loads typically experienced in the posterior region of the spine, for example, up to about 80 shore A. In other words, the spacer is preferably capable of resisting compressive motion (or loads) with a stiffness of about 40 to about 240 N/mm (newtons per millimeter). The material is preferably injectable, and may be compressible immediately or after a time, for example, after curing. For purposes of the invention, the compressibility characteristic is necessary during end use, i.e., after implantation. Materials that could be used include, for example, a plurality of beads (e.g., polymer beads) that in the aggregate are compressible, or materials that change state from exhibiting fluid properties to exhibiting properties of a solid or semi-solid. Examples of such state-changing materials include two-part curing polymers and adhesive, for example, platinum-catalyzed silicone, epoxy, polyurethane, etc.

As noted above, the structure provides support for and containment of the container when filled. The structure comprises, for example, a structural mesh comprising a plurality of fibers 408. For example, the structure can take the form of a fabric jacket, as shown in FIG. 4. The structure, a fabric jacket in this example, also contains and helps protect the container from bulging and damage from forces external to the container. The fibers comprise, for example, PET fabric, polypropylene fabric, polyethylene fabric, and/or steel, titanium or other metal wire. Depending on the application, the structure may be permeable in some respect. For example, if bone or tissue growth is desired to attach to the structure, permeability to the tissue or bone of interest would be appropriate. As another example, permeability of the structure may be desired to allow the material used to fill the container to evacuate air or water, for example, from the container, in order to prevent bubbles from forming inside. Where a mesh is used, for example, the degree of permeability desired can be achieved by loosening or tightening the weave.

Although the structure is shown in its final, roughly H-shape in the example of FIG. 4, it will be understood that in practice, the structure can be made to be folded (e.g., a fabric mesh) and/or unexpanded. Further, the structure can have a shape other than that shown. A folded or unexpanded state facilitates implantation, allowing for a smaller surgical opening, and unfolding or expansion in situ upon filling of the container.

One example of the construction of a fabric jacket 700 for use as one example of a structure of the present invention will now be described with reference to FIG. 7. Two roughly cylindrical fabric members 702 and 704 are sewn together around a periphery 706 of an opening along a side (not shown) in each. An opening 708 is created in one of the members for accepting the container, for example, by laser cut. In one example, a conduit described above would poke through opening 708. The ends of the cylindrical members (e.g., end 710) are then trimmed and sewn shut, as shown in broken lines (e.g., lines 712) in FIG. 7.

FIG. 5 depicts an outer view of another example of a surgical spacer 500 in accordance with the present invention. A container conduit 501 is shown pointing outward from an opening 503. As shown, the structure 502 limits the expansion of the spacer and may create a rough final shape, in this example, a rough H-shape. The structure comprises a fabric jacket 504, similar to that described above with respect to FIG. 4, that is soaked through with a dispersion polymer 506 (e.g., silicone). The dispersion polymer (after curing) acts as the container and is shown filled in FIG. 5. This is one example of the container and the structure being integral.

FIG. 6 is a cross-sectional view of another example of a surgical spacer 600 in accordance with the present invention. Surgical spacer 600 is similar to the spacer of FIG. 5, except that instead of being soaked in a dispersion polymer, a fabric jacket 602 is coated with a dispersion polymer (e.g., silicone) 604 or other curable liquid appropriate for the container material, creating an outer container. The coating can be done in a conventional manner, for example, by dip molding on the outside of the fabric jacket.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims.

What is claimed is:

1. A surgical spacer, comprising:
a flexible container for containing a material that is compressible during end use, wherein the container is substantially impermeable to the material; and
a structure for at least part of the container when containing the material.

2. The surgical spacer of claim 1, wherein the material is flowable during filling of the container, the surgical spacer further comprising a conduit coupled to the container for accepting the material.

3. The surgical spacer of claim 2, wherein the conduit comprises a one-way valve.

4. The surgical spacer of claim 1, wherein the container is situated inside the structure.

5. The surgical spacer of claim 1, wherein the container is situated outside the structure.

6. The surgical spacer of claim 1, wherein the container is integral with the structure.

7. The surgical spacer of claim 1, wherein the container comprises a silicone copolymer.

8. The surgical spacer of claim 7, wherein the silicone copolymer comprises silicone.

9. The surgical spacer of claim 1, wherein the container comprises rubber.

10. The surgical spacer of claim 1, wherein the container comprises polyurethane.

11. The surgical spacer of claim 1, wherein the container comprises polyethylene terephthalate (PET).

12. The surgical spacer of claim 1, wherein the container comprises polyolefin.

13. The surgical spacer of claim 1, wherein the container comprises polycarbonate urethane.

14. The surgical spacer of claim 1, wherein the material comprises a curable polymer.

15. The surgical spacer of claim 1, wherein the material comprises an adhesive.

16. The surgical spacer of claim 1, wherein the structure comprises a structural mesh.

17. The surgical spacer of claim 16, wherein the structural mesh comprises PET fabric.

18. The surgical spacer of claim 16, wherein the structural mesh comprises polypropylene fabric.

19. The surgical spacer of claim 16, wherein the structural mesh comprises polyethylene fabric.

20. The surgical spacer of claim 16, wherein the structural mesh comprises metal wire.

21. The surgical spacer of claim 20, wherein the metal wire comprises steel wire.

22. The surgical spacer of claim 20, wherein the metal wire comprises titanium.

23. The surgical spacer of claim 1, wherein the surgical spacer comprises an interspinous spacer, and wherein the structure is shaped to fit between adjacent spinous processes.

24. The surgical spacer of claim 23, wherein the surgical spacer is capable of resisting a compressive load with a stiffness of about 40 N/mm to about 240 N/mm.

25. The surgical spacer of claim 1, wherein the structure is at least partially permeable.

26. The surgical spacer of claim 1, wherein the surgical spacer is shaped to replace at least part of an intervertebral disc.

27. A method of surgically spacing adjacent body parts, the method comprising:
providing a surgical spacer, comprising:
a flexible container for containing a material that is compressible during end use, wherein the container is fillable and substantially impermeable to the material; and
a structure for at least part of the container when containing the material;
implanting the surgical spacer between adjacent body parts; and
filling the container with the material.

28. The method of claim 27, wherein the surgical spacer further comprises a one-way valve coupled to the container for accepting the material, and wherein the filling comprises injecting the material into the container through the one-way valve.

29. The method of claim 27, wherein the surgical spacer comprises an interspinous spacer, and wherein the implanting comprises implanting the interspinous spacer between adjacent spinous processes.

30. An interspinous spacer, comprising:
a flexible container for containing an injectable curable material that is compressible during end use, wherein the container is substantially impermeable to the injectable curable material;
a structural mesh for at least part of the container when containing the injectable curable material, wherein the structural mesh is shaped to fit between adjacent spinous processes; and
a conduit coupled to the container for accepting the injectable curable material.

31. The interspinous spacer of claim 30, wherein the container comprises at least one of a silicone copolymer, rubber, polyurethane, PET, polyolefin and polycarbonate urethane.

32. The interspinous spacer of claim 30, wherein the container is situated inside the structural mesh.

33. The interspinous spacer of claim 30, wherein the injectable curable material comprises a silicone copolymer.

34. The interspinous spacer of claim 30, wherein the structural mesh comprises at least one of a PET fabric, polypropylene fabric, polyethylene fabric and metal wire.

35. The interspinous spacer of claim 30, wherein the conduit comprises a one-way valve.

36. A method of spacing adjacent spinous processes, the method comprising:
providing an interspinous spacer, comprising:
a flexible container for containing an injectable curable material that is compressible during end use, wherein the container is substantially impermeable to the injectable curable material;
a structural mesh for at least part of the container when containing the injectable curable material, wherein the structural mesh is shaped to fit between adjacent spinous processes; and
a valve coupled to the container for accepting the injectable curable material;
implanting the interspinous spacer between adjacent spinous processes; and
injecting the injectable curable material into the container through the valve.
